(19) United States
(12) Plant Patent Application Publication
(10) Pub. No.: US 2014/0345027 P1
(43) Pub. Date: Nov. 20, 2014

(54) Salvia plant named 'Eggben007'

(50) ;

(71) Applicant: Howard Bentley, Wonga Park (AU)

(72) Inventor: Howard Bentley, Wonga Park (AU)

(21) Appl. No.: 13/986,590

(22) Filed: May 15, 2013

(51) Int. Cl. A01H 5/00

(52) U.S. Cl. PLT/475

(57) ABSTRACT

A new and distinct Salvia cultivar named ‘EGGBEN007’ is disclosed, characterized by a dense, bushy and upright plant habit, yellow colored flowers that are flushed with pink. The new variety is a Salvia, normally produced as an outdoor garden or container plant.

### LATIN NAME OF THE GENUS AND SPECIES

Salvia gregii×microphylla

### VARIETY DENOMINATION

‘EGGBEN007’

### BACKGROUND OF THE INVENTION

The new Salvia cultivar is a product of a planned breeding program conducted by the inventor, Steve Eggleton, in Wonga Park, Victoria, Australia. The objective of the breeding program was to produce new Salvia varieties with denser plant habits, strong garden performance, in a range of flower colors. The controlled pollination resulting in this new variety was made during March and April of 2008.

The parent variety is Salvia gregii×microphylla ‘Eggben005’, U.S. Plant patent application Ser. No. 13/506,155. The parent variety was self-pollinated to produce an F2 generation of seed. This seed was collected, sown and grown outdoors under field conditions. The new variety was identified as a potentially interesting selection in March of 2010, at a research greenhouse in Wonga Park, Australia. Further observations and evaluations were made throughout 2010 and 2011.

Asexual reproduction of the new cultivar ‘EGGBEN007’ by vegetative cuttings was first performed during 2010, at a research greenhouse in Wonga Park, Australia. Subsequent propagation has shown that the unique features of this cultivar are stable and reproduced true to type on successive generations.

### SUMMARY OF THE INVENTION

The cultivar ‘EGGBEN007’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘EGGBEN007’ These characteristics in combination distinguish ‘EGGBEN007’ as a new and distinct Salvia cultivar:

1. Upright to bushy plant habit.

2. Yellow flower petals with a pink margin

3. Corolla lower lip predominately yellow.

4. Dense plant habit.

### PARENT COMPARISON

Plants of the new cultivar ‘EGGBEN007’ are similar to plants of the parent, in most horticultural characteristics, however, plants of the new cultivar ‘EGGBEN007’ are more upright in plant habit. Additionally, the new variety typically shows strong anthocyanin coloration in the stems, and has a different color corolla.

### COMMERCIAL COMPARISON

Plants of the new cultivar are best compared to the commercial variety Salvia gregii×microphlla ‘EGGBEN003’, U.S. Plant patent application Ser. No. 13/506,161. The two Salvia varieties are very similar in most horticultural characteristics, however, the new variety produces a lower lip colored yellow and flushed with pink, whereas the comparator has a cream colored lower lip. Foliage differs as well; the new variety has more deeply incised foliage than ‘EGGBEN003’.

Plants of the new cultivar can also be compared to the commercial variety Salvia jamensis ‘Moonlight Serenade’, unpatented. The two Salvia varieties are very similar in many characteristics, however, the new variety produces a lower lip colored yellow and flushed with pink, whereas the comparator has a cream colored lower lip. Plants of the new variety are significantly denser than plants of ‘Midnight Serenade’.

### BRIEF DESCRIPTION OF THE PHOTOGRAPHS

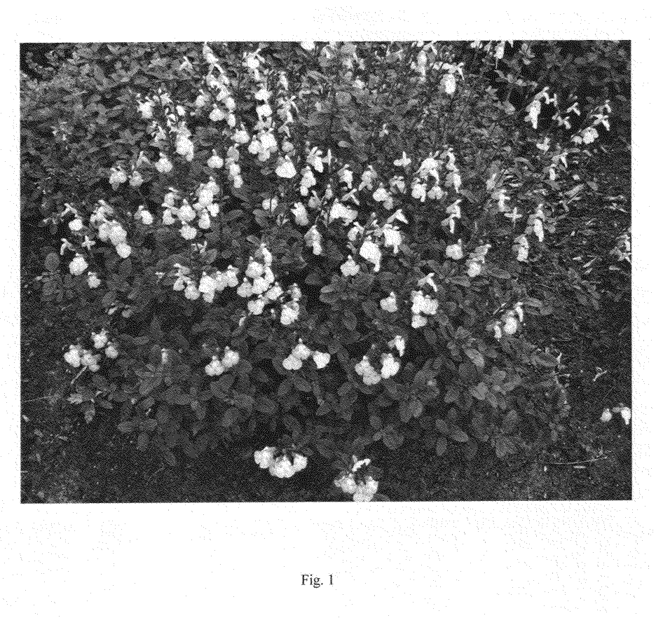

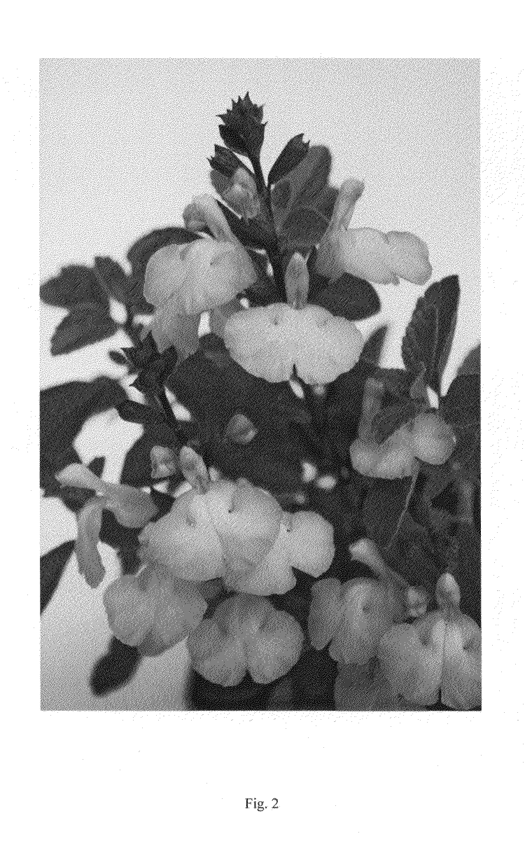

The accompanying photograph in FIG. 1 illustrates in full color a typical plant of ‘EGGBEN007’. FIG. 2 illustrates in full color typical flowers of ‘EGGBEN007’. The plants was grown outdoors, in Wonga Park, Australia. Age of the plant photographed is approximately 24 weeks from an unrooted cutting. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘EGGBEN007’ plants grown outdoors in Wonga Park, Australia. The growing temperature ranged from approximately 5° C. to 35° C. Measurements and numerical values represent averages of typical plant types.
Botanical classification: Salvia gregii×microphylla ‘EGGBEN007’
Propagation:
   Time to initiate roots.—About 5-8 days at approximately 25° C.
   Root description.—Fine, densely fibrous.
   Time to produce a rooted cutting.—About 21 days at 25° C.
Plant:
   Growth habit.—Upright, bushy annual.
   Height.—Approximately 30 cm to top of foliage. Approximately 40 cm to top of flowering plane. Measured from soil level of pot.
   Plant spread.—Approximately 24 cm.
   Growth rate.—Rapid.
   Branching characteristics.—Free-branching, Approximately 8 to 10 primary branches.
   Length of primary lateral branches.—Approximately 35 cm.
   Diameter of lateral branches.—Approximately 0.35 cm.
   Quantity of lateral branches.—About 12 to 18.
   Stem.—Color: Near RHS Green 138A, flushed with Greyed-Purple 183B Pubescence: Minute pubescence.
   Internode length.—Approximately 2.0 to 3.0 cm.
   Age of plant described.—Approximately 24 weeks from an unrooted cutting.
Foliage:
   Leaf.—Arrangement: Opposite. Quantity: Approximately 16 fully expanded per main branch. Average Length: Approximately 4.9 cm., including petiole. Average Width: Approximately 2.3 cm. Shape of blade: Obovate. Apex: Obtuse. Base: Obtuse to nearly truncate. Attachment: Stalked. Margin: Crenate. Texture of top surface: Matte, minute, coarse pubescence. Texture of bottom surface: Matte, minute, coarse pubescence. Color: Young foliage upper side: Near RHS Green 137A. Young foliage under side: Near RHS Green 137B. Mature foliage upper side: Near RHS Green 137A. Mature foliage under side: Near RHS Green 138A. Venation: Type: Pinnate. Venation color upper side: Near RHS Yellow-Green 144A. Venation color under side: Near RHS Green 143C. Petiole: Average Length: Approximately 0.8 cm. Diameter: Approximately 0.1 cm. Color: Near Green 143C.
Flower:
   Natural flowering season.—Flowering from early Spring through late Summer.
   Inflorescence type and habit.—Flowers arranged in verticillasters on spikes.
   Flower longevity on plant.—Individual flowers last approximately 1 week on the plant. Each spike lasts approximately 7 weeks with flowers.
   Quantity of flowers.—About 6 to 8 buds and 4 to 5 fully opened flowers per spike, at one time. Mature plants have approximately 10 to 20 spikes.
   Spike size.—Diameter: Approximately 5.8 cm. Height: Approximately 7.5 cm.
   Individual flowers.—.
   Size.—Diameter: Approximately 2.5 cm. Length: Approximately 2.9 cm. Persistence: Non-persistent. Fragrance: Moderate, typical Salvia scent.
   Corolla.—Petal arrangement: The corolla is sympetalous and typically bilabiate with 2 small, highly fused lobes forming an upper lip and 3 larger highly fused lobes forming a lower lip Margin: Nearly entire, slightly scalloped Tip shape: Upper lip tip retuse, lower lip tip rounded with scallops. Length: Upper lip Approximately 1.2 cm, lower lip Approximately 1.7 cm. Tube length Approximately 2.0 cm. Width: Upper lip Approximately 0.4 cm, lower lip Approximately 2.4 cm. Tube width Approximately 0.8 cm. Texture: Upper lip: Highly pubescent on exterior surface. Interior surface glabrous Lower lip: Glabrous all surfaces.
   Color.—Upper Lip.
   When opening.—Inner surface: Background color Near RHS Yellow 11C, strong flush near Red 43C throughout, and completely covering margin. Outer surface: Background color Near RHS Yellow 11C, strong flush near Red 43C throughout, and completely covering margin.
   Fully opened.—Inner surface: Background color Near RHS Yellow 13D, moderate flush near Red 43C throughout, and strongest along margin. Outer surface: Background color Near RHS Yellow 13D, faint flush near Red 43C throughout, and strongest along margin.
   Fading.—Inner surface: Near RHS Yellow 13D, faint flush near Red 43C throughout, and strongest along margin. Outer surface: Near RHS Yellow 13D, faint flush near Red 43C throughout, and strongest along margin.
   Color.—Lower Lip.
   When opening.—Inner surface: Background color Near RHS Yellow 11C, strong flush near Red 43C throughout, and completely covering margin. Outer surface: Background color Near RHS Yellow 11C, strong flush near Red 43C throughout, and completely covering margin.
   Fully opened.—Inner surface: Background color Near RHS Yellow 13D, moderate flush near Red 43C throughout, and strongest along margin. Outer surface: Background color Near RHS Yellow 13D, faint flush near Red 43C throughout, and strongest along margin.
   Fading.—Inner surface: Near RHS Yellow 13D, faint flush near Red 43C throughout, and strongest along margin. Outer surface: Near RHS Yellow 13D, faint flush near Red 43C throughout, and strongest along margin.
   Color.—Tube.
   When opening.—Inner surface: Near RHS Yellow 13D, faint flush near Red 43D Outer surface: Near RHS Yellow 13D, faint flush near Red 43D.
   Fully opened.—Inner surface: Near RHS Yellow 13D, faint flush near Red 43D Outer surface: Near RHS Yellow 13D, faint flush near Red 43C.
   Fading.—Inner surface: Near RHS Yellow 13D, faint flush near Red 43D Outer surface: Near RHS Yellow 13D, faint flush near Red 43D.
   Bud.—Shape: Ovoid Length: Approximately 1.4 cm Diameter: Approximately 0.8 cm Color: Near RHS Red 43C.
   Calyx.—Length: Approximately 1.4 cm Diameter: Approximately 0.9 cm Shape: Tubular.
   Sepals.—Shape: Quantity per flower: 3, fused to form a tube. Unfused Apex: Length: Approximately 0.6 cm Width: Approximately 0.6 cm Margin: Entire Apex: Acute. Texture: Pubescent. Color: Immature: Near RHS Green 138A, flushed Greyed-Orange 166A Mature: Near RHS Green 138A with very faint flush near Greyed-Purple 187A.
   Peduncle.—Length: Average 2.7 cm. Diameter: 0.3 cm Color: Near RHS Green 137B, flush near Greyed-Purple 187A Orientation: Upright, straight. Strength: Strong. Texture: Pubescent.
   Pedicels.—Length: Average 0.2 cm. Diameter: 0.1 cm Color: Near RHS Greyed-Purple 187A Orientation: Straight, approximately 45 degree angle from attachment. Strength: Flexible. Texture: Pubescent.
Reproductive organs:
   Stamens.—Number: 2 Filament length: Approximately 1.3, approximately 0.6 cm of length fused to petal. Filament color: Near RHS White N155B.
   Anthers.—Shape: Very narrowly elliptic Length: Approximately 3 mm Color: Near RHS Greyed-Yellow 161C. Pollen: Moderate colored near Yellow-Orange 23B.
   Pistil.—Number: 1 Length: Approximately 2.7 cm Color: Near RHS White N155B Stigma: Shape: Linear, curled. Color: Near RHS Red-Purple 61A Ovary color: Near RHS Green-Yellow 1B.
Other characteristics:
Seeds and fruits: Not observed to date.
Disease/pest resistance: Neither resistance nor susceptibility to pathogens and pests common to Salvia have been observed.
Temperature tolerance: The new variety tolerates temperatures between 5 to 35° C.

What is claimed is:

1. A new and distinct cultivar of Salvia plant named ‘EGGBEN007’ as herein illustrated and described.
